(19) United States
(12) Patent Application Publication
(10) Pub. No.: US 2022/0008763 A1
(43) Pub. Date: Jan. 13, 2022

(54) SMART FACE PROTECTIVE DEVICE AND SYSTEM FOR INFECTION CONTROL

(71) Applicant: Ahmad Saleh, Dubai (AE)

(72) Inventors: Ahmad SALEH, Dubai (AE); Ryan Saleh, Dubai (AE)

(21) Appl. No.: 17/373,226

(22) Filed: Jul. 12, 2021

Related U.S. Application Data
(60) Provisional application No. 63/050,848, filed on Jul. 12, 2020.

(51) Int. Cl. A62B 18/08; H04W 4/38; A41D 13/11; A62B 18/04; A61F 9/02

(52) U.S. Cl. CPC A62B 18/08; H04W 4/38; A61F 9/029; A62B 18/04; A41D 13/11

(57) ABSTRACT

There is provided a smart face protective device, preferably as part of a helmet or another head held device, to be used by a user for preventing or reducing spread of infectious droplets through a portal portion of the user's face, where the face protective device is configured to be automatically adjusted while being in use between a secured position and an unsecured position based on an electric signal associated to a monitored health and safety condition of the user. There is also provided a system, a network and a method for allocating smart face protective devices to members of a community and managing these devices and data collected thereby for preventing or reducing risks of virus contamination and spread, namely COVID-19, between the members of the community.

### CROSS-REFERENCE TO RELATED APPLICATIONS AND PRIORITY

This patent application claims priority from U.S. Provisional Patent Application No. filed 63/050,848. This patent application is herein incorporated by reference in its entirety.

### FIELD OF THE INVENTION

The present invention relates to personal protective equipment, and more particularly to a smart face protective device for wear or use by a user for blocking splashes and sprays between the user and others and for preventing hands from reaching the user' s face in order to prevent or reduce the spread of infectious diseases such as COVID-19, as well as a smart face protective system and network for managing a group of smart face protective devices worn/used by a group of users, the device being of a particular utility for institutions such as schools, hospitals, and elderly houses, as well as for crowded areas such as airports and shopping malls.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Hundreds of million of students from prekindergarten through primary and secondary grades globally have had their education disrupted by school closures amid the continuing spread of a new coronavirus COVID-19, a figure the U.N. agency dealing with the crisis says is unprecedented. There are school closures of some kind in dozens of countries on three continents with hundreds of millions of students around the world facing upheaval, including many countries that have shut schools nationwide. The United Nations has warned of the unparalleled scale and speed of the educational disruption being caused by coronavirus.

Schools are starting to take precautions to prevent virus outbreaks, including canceling trips, teaching students to wash their hands for an appropriate period of time with soapy water and sending reassuring messages to parents. A few schools have closed, and others are considering doing that if necessary.

Other schools have implemented the wearing of face shields or masks as mandatory precautionary measures. Face shields are simple, transparent screens that cover the face and help prevent infectious droplets from entering the eyes, nose and mouth. They are usually worn for blocking splashes and sprays from reaching the face and making it preventing workers from touching their faces.

The wear of traditional face shields is less effective for children and people with special needs (even elderly) as they normally are not comfortable in keeping their face shields or masks on covering their faces for long hours and they normally don't have the maturity/judgment to open and close their face shields/masks when the health/safety situation requires it. The wear of traditional face shields/masks is also not convenient in all conditions and environments.

Schools, hospitals and other institutions have been facing increasing challenging of controlling and managing the wear of masks and face shields within their institutions, in addition to the challenges for taking precautionary measures as well as corrective measures for enhancing the safety and health conditions of their members.

### SUMMARY OF THE INVENTION

As an aspect of the invention, there is provided a personal protective device or equipment (also called a smart face protective device or SFPD) which is configured to be used by a user for preventing or reducing spread of infectious droplets and body fluids through a portal portion of the user's face, where the face protective device is configured to be automatically adjusted while being in use between a secured position and an unsecured position based on an electric signal associated to a monitored health and safety condition of the user.

The personal protective device is configured to be worn or used by the user and to automatically alternate between a secured and unsecured positions (close and open the face shield respectively, for example) based on the health and safety conditions of the user or persons passing in proximity of the user. This is as an infant (such as children at school for example) might not have the judgment and/or the speed and/or the physical capability to close the face shield when another person is passing in proximity or in case he has a high temperature or in case there is any other health/safety condition putting him or other persons at risk. At the same time, it will be difficult to convince an infant to cover his/her face using a helmet or a face shield or a mask for a long period of time as this becomes uncomfortable and impractical for them to breathe and communicate with others.

Also, a person in special needs, such as a patient at a hospital or a handicapped person on a wheel chair or an elder person in an elderly house) might lack the judgment and/or the physical condition to open and close the face shield and might feel uncomfortable or impractical to have the face shield/mask covered during long hours.

The personal protective device is also adapted to be used in crowded areas such as airports and shopping malls with many persons passing across which makes it impossible to be able to manually monitor passing persons and to put on the mask or face shield when required. The personal protective device provides a 360 degrees monitoring capabilities (from behind, in front, from the sides) adapted to spot intruding persons coming in physical proximity of the user breaching the minimum social distance threshold. The personal protective device is capable of reacting automatically to any such intrusion to put the face shield in the secured positon to block any fluid communication between the user and the intruding persons.

In an embodiment of the invention, the secured position comprises a position in which the portal portion of the user's face is isolated from the external environment for blocking spread of splashes and sprays and other body fluids between the user and others through said portal portion and for blocking the user's hands from reaching to said portal portion of the face. In an embodiment of the invention, the portal portion of the face comprises the eyes, nose and mouth which are the mucous membranes of the face through which droplets and body fluids can pass to the inside of the body and spread infections.

In an embodiment of the invention, the unsecured position comprises a position in which at least a part of the portal portion is not in a secured position. For example, if the face shield is open and the eyes, nose and/or mouth of the user are exposed to the external environment such as infectious droplets, splashes and/or other fluids can be communicated between the user and other persons through these membranes, then the face shield is considered to be in an unsecured position.

In an embodiment of the invention, the smart face protective device is designed to replace a traditional face shield which requires manually covering and uncovering the face when required which can be inefficient, inconvenient, impractical or impossible for certain people and for certain situations. For example, traditional face shields and masks requiring the manual wearing of these are impractical for kids at kindergartens and schools.

In an embodiment of the invention, the smart face protective device is configured for young children and kids who might not have the required maturity or attention to cover their faces with a mask or a face shield when required and who are uncomfortable in keeping on a face shield covering their face during a long period of time. A kid cannot be trusted to keep on a mask or a face shield during long period of time, where the importance of providing a smart device designed to automatically activate and deactivate (close and open) a face shield based on health and safety conditions. When the device detects a risk of virus contamination or another predefined condition related to health/safety, the device automatically activates the face shield (by closing the face shield) to keep the kid safe. When the risk is removed, the device automatically deactivates the face shield (by opening the face shield) to keep the kid comfortable.

In an embodiment of the invention, the smart face protective device is also designed for people with special needs and elderlies who do not have the required reflexes, physical or mental capabilities and/or judgments to wear a mask or to cover their faces with a face shield when required.

In an embodiment of the invention, the smart face protective device (SFPD) comprises a controller (or a microprocessor or an electrical or electronic circuit) in communication with a Health & Safety Monitoring Unit for receiving the electric signal associated to the health and safety condition of the user in real time while the Health & Safety Monitoring Unit is in operation for monitoring the health and safety condition of the user. The controller receives the electric signal from the Health & Safety Monitoring Unit and automatically adjusts the face protective device in the secured or unsecured position as a function of the electric signal received. For example, when/if the electric signal received from the Health & Safety Monitoring Unit is indicative of a health/safety risk of the user or other persons, then the controller puts the face protective device in the secured position (by closing the face shield for example). From another side, when/if the electric signal received from the Health & Safety Monitoring Unit indicates absence of risk related to health/safety, then controller puts or allows or enables the face protective device to be in the unsecured position (by automatically opening or allowing the face shield to be opened by the user for example).

In an embodiment of the invention, the Health & Safety Monitoring Unit comprises a thermal detector for monitoring a body temperature of the user and the controller is configured to automatically adjust the face protective device in the secured position when the body temperature of the user is above a predefined temperature threshold (for example 37.8 or 38 degrees Celsius). This is as a high temperature of the user might be indicative of the user being sick or being infected by a contagious disease (such as COVID-19), and in this case the face protective device is automatically adjusted into the secured position to isolate the user's face from the external environment in order to prevent/reduce the risks of the user contaminating others persons.

In an embodiment of the invention, the face protective device comprises a notification unit in electrical communication with the controller configured to notify a health/safety risk condition such as a high temperature above the predefined temperature threshold. The notification unit is configured to notify the user or other users through a user interface such as a screen, a led, a voice message, or a vibrator. In an embodiment of the invention, the notification unit is configured to be connected to a remote administration device through a data network (such as WIFI) to notify an administrator (such as a teacher or school director) of presence of a health/safety risk associated to the user.

In an embodiment of the invention, the thermal detector is configured to monitor the body temperature of others persons passing or being in proximity of the user (also called intruding person or intruding persons) and the controller is configured to automatically adjust the face protective device in the secured position when a body temperature of the intruding person is detected to be above the predefined temperature threshold (for example 37.8 or 38 degrees Celsius). This is as a high temperature of the intruding person might be indicative of the intruding person being sick or having been infected by a contagious disease (such as COVID-19), and in this case the face protective device is automatically adjusted into a secured position to isolate the user's face in order to prevent/reduce the risks of the intruding person infecting the user.

In an embodiment of the invention, the thermal detector monitors the body temperature of intruding persons being within a predefined social safety distance threshold which is preferably around 6 feet (however this can vary depending on the application). This is persons outside this social safety distance do not represent a risk on the user, and therefore there is no need in obtaining their temperature.

In an embodiment of the invention, the intruding persons are other users (for example other kids at the same school) having similar face protective devices, and wherein each one of the face protective devices comprises an inter-device communication unit in communication with the controller and with a memory. In an embodiment of the invention, the inter-device communication units associated to the various face protective devices are configured to exchange health/safety data relative to their respective users (for example the identity of the intruding persons passing within the social safety distance threshold, and the temperature of the intruding users) and to store this data inside the memory of their respective SFPD devices.

In an embodiment of the invention, the data exchanged by a face protective device comprises an indication of any health/safety risk associated to its respective user (for example an indication of high temperature of the user, or a measure of the temperature itself), and wherein the face protective device automatically adjusts the face protective device in the secured position (for example by closing the face shield) when/if the temperature of the intruding person as obtained by the inter-communication unit is above the predefined temperature threshold. Preferably, the identity of the intruding user (or associated face protective device) is stored inside the memory of the user's face protective device which can be retrieved at any future time for tracking and identification purposes of infected persons.

In an embodiment of the invention, the face protective device is designed to reduce the risk of infection by the COVID-19, and the predefined temperature threshold is set around 38 degrees Celsius. Preferably, the predefined temperature threshold is in the range of 37.5 degrees Celsius and 38.5 degrees Celsius. A temperature above this predefined temperature threshold is indicative of the presence of a fever which might be indicative of the presence of a virus such as COVID-19.

In an embodiment of the invention, the face protective device comprises a user interface having an input/output interface configured to enable a user to set and change the predefined temperature threshold and/or the social safety distance threshold depending on the application and requirements.

In an embodiment of the invention, the thermal detector comprises a non-contact infrared sensor configured to detect the temperature of the user. In an embodiment of the invention, the non-contract infrared sensor is also configured to detect the temperature of intruding persons passing in proximity of the user (preferably those who are within the social safety distance threshold only).

In an embodiment of the invention, the Health & Safety Monitoring Unit further comprises a social distance detector for detecting presence of an intruding person within a predefined minimum social safety distance threshold of the user, and wherein the controller is configured to automatically adjust the face protective device in the secured position when the intruding person is detected within the predefined minimum social safety distance threshold. Preferably, the inter-device communication unit obtains the identity of the intruding person (or associated device) and stores the information inside the memory.

In an embodiment of the invention, the social safety distance threshold is around 6 feet. This is the minimum distance required between two persons in order to prevent/reduce the risks of communicating droplets and body fluids between the two persons and therefore being contaminated by COVID-19 or other infections. This minimum threshold can preferably be set and changed depending on the application and requirements using the input/output interface of the face protective device.

In an embodiment of the invention, the face protective device comprises a face shield unit comprising a face shield configured to be automatically closed/activated for securely shielding the portal portion of the user's face when the face protective device is in the secured position, and to be open/deactivated when the face protective device is in the unsecured positon. According to this embodiment, a face shield is used to act as a barrier between the user's portal portion of the face and the external environment.

The face shield is made of a suitable material depending on the application. In an embodiment of the invention, the face shield is made of a solid material such as transparent plastic or glass acting as barrier to droplets and body fluids without obstructing the vision of the user.

In an embodiment of the invention, the face shield comprises an ultra-violet (UV) light configured to be automatically activated and deactivated to respectively secure and unsecure the face protective device depending on the health/safety condition of the user. In another embodiment of the invention, the face shield comprises an air ionizer configured to create an ionized layer of air acting as barrier to infected droplets and body fluids.

In an embodiment of the invention, the face protective device is configured to be used in an indoor environment for preventing or reducing the spread of COVID-19. The indoor environment can be any type of organization or institution or establishment such as an education institution, an elderly care center, a hospital or a shopping mall. In an embodiment of the invention, the face protective device is particularly useful for children in the age range of 2 to 15 years old.

In an embodiment of the invention, the face protective device has a unique identifier allowing to uniquely identify the face protective device. In an embodiment of the invention, the face protective device is associated to a specific user and the unique identifier enables to uniquely identify the user associated to the face protective device. In an embodiment of the invention, the unique identifier is stored inside the memory of the face protective device and accessed to/used by the controller depending on the application. The unique identifier can be static or dynamic such that it can be updated to reflect the identity of the user using the face protective device at any given time. In an embodiment of the invention, the input/output interface of the face protective device is configured to enable the set up/change of the unique identifier based on the identity of the user using the device.

In an embodiment of the invention, the Health & Safety Monitoring Unit is an external Health & Safety Monitoring Unit located remote from the smart face protective device. In this case, the communication between the controller and the Health & Safety Monitoring Unit is a remote wireless communication (through a data network such as WIFI or Bluetooth or any other suitable wireless communication technology) conducted through the communication unit of the face protective device.

hi an embodiment of the invention, the Health & Safety Monitoring Unit comprises an identification system configured to identify the user. The identification system can for example be an RFID system wherein the RFID reader can be located at the remote (external) Health & Safety Monitoring Unit and the RFID tag is located at the face protective device. The Health & Safety Monitoring Unit identifies the user, monitors his body temperature and sends a signal to the face protective device of the user (through the controller) in order to automatically adjust the shield in the secured position when the temperature of the user is beyond the predefined temperature threshold. In another embodiment, the thermal detector of the Health & Safety Monitoring Unit screens the body temperature of other persons passing or being located in proximity of the user (the intruding persons), and sends a signal to the face protective device of the user (through the communication unit connected to the controller) in order to automatically adjust the shield in the secured position when the temperature of an intruding person is beyond the predefined temperature threshold.

In an embodiment of the invention, the Health & Safety Monitoring Unit is mechanically coupled to the smart face protective device.

In an embodiment of the invention, the smart face protective device is a device configured to be mechanically coupled or affixed to a functional article such as a furniture. For example, the functional article can be a cubicle, a desk, a bed, a wheelchair, an internal component of a vehicle, etc.

In an embodiment of the invention, the face protective device comprises a face shield unit comprising a face shield providing a safety/security barrier to the user from the external environment and intruding persons by automatically adjusting the face shield between the secured and unsecured positions based on safety/health conditions monitored using the Health & Safety Monitoring Device. In one example, the face shield is a physical shield made of a solid material (such as a plastic or glass window for example). The face shield can be mounted along a peripheral portion of the functional article (such as a student cubicle/desk or a wheelchair or a patient bed) and configured to provide a security zone to the user (school student or patient) and protect him/her form infected intruding persons (or the opposite, protecting the other persons from an infected user).

In an embodiment, the face shield unit comprises a motor for opening and closing the face shield (window) based on the safety/health conditions as monitored by the Health & Safety Monitoring Unit (the monitoring and the adjustment of the face shield window being conducted in real time). In another embodiment, the face shield unit comprises a UV light source configured to activate and deactivate the UV light based on the safety/health conditions as monitored by the Health & Safety Monitoring Unit (the monitoring and the adjustment of the face shield window being conducted in real time).

As an example, the face protective device can be fixed to cubicles/desks of school kids at school such that each cubicle/desk is equipped with a corresponding face protective device for providing school kids with a safe environment for them and others while studying. Each kid student in this case can have his own safe cubicle/desk environment with the minimum intrusion possible where the face shield is only activated (for example the shield window closes) only when required (for example when there is another intruding kid approaching the cubicle/desk or when the kid himself has some symptoms of infection such as high temperature). As another example, the face protective device can be fixed to patients' beds in hospitals such that each patient bed is equipped with a corresponding face protective device for providing patients with a safe environment for them and others while being hospitalized. Each patient in this case can have his own safe bed environment with the minimum intrusion possible where the face shield is only activated (for example the shield window closes) only when required (for example when there is another intruding patient or healthcare professional approaching or when the patient himself has some symptoms of infection such as high temperature). Another example, the face protective device can be fixed to wheelchairs for providing persons with special needs with a safe environment for them and others while commuting.

In an embodiment of the invention, the face protective device is a portable device configured to be carried or worn by the user, such as for example a smart helmet or a smart face shield. In this case, the face shield unit of the face protective device comprises a face shield preferably made of solid material and, the face shield unit further comprises a motor in connection with the controller configured to open/close the face shield based on the instructions received from the controller which are based on the electric signals received from the Health & Safety Monitoring Unit.

In an embodiment of the invention, the face protective device comprises a mounting structure configured to be carried or worn by the user, and a transparent face shield coupled to the mounting structure and configured to be automatically adjusted in the secured and unsecured positions.

As a further aspect of the invention, there is provided a smart helmet device to be worn by a user for preventing or reducing risks of spreading infectious droplets through a portal portion of the user's face, the smart helmet device comprising:
a face shield unit comprising a face shield;
a Health & Safety Monitoring Unit for monitoring a health and safety condition of a user and outputting an electric signal with an indication of the health and safety condition of the user;
a controller in communication with the Health & Safety Monitoring Unit and the face shield unit for receiving the electric signal associated to the monitored health and safety condition of the user and automatically adjusting the face shield between a secured position and an unsecured position based thereon.

In an embodiment of the invention, the secured position comprises a position in which the portal portion of the user's face is isolated from the external environment for blocking spread of splashes and sprays between the user and others through said portal portion and for blocking the user's hands from reaching to said portal portion of the face. In an embodiment of the invention, the unsecured position comprises a position in which at least a part of the portal portion is not in a secured position. In an embodiment of the invention, the portal portion of the face comprises the eyes, nose and mouth.

In an embodiment of the invention, adjusting the face shield into a secured position comprises closing the face shield such that the user's face (or at least the portal portion) is protected and isolated from the external environment to avoid any fluid communication of droplets or splashes with other persons and to restrict the user's hands of reaching to the face (or at least the portal portion of the face). In an embodiment of the invention, adjusting the face shield into an unsecured position comprises opening the face shield such that the face of the user is exposed to the external environment.

Preferably, the face shield unit comprises a motor (preferably an electric motor) connected to the face shield for opening and closing the face shield (or at least a portion of the face shield responsible of protecting the portal portion of the user's face) as a function of the electric signal received from the Health & Safety Monitoring Unit.

In an embodiment of the invention, the Health & Safety Monitoring Unit comprises a thermal detector for monitoring a body temperature of the user, and wherein the controller is configured to put the face shield in the secured position (ex. closing the face shield) when the body temperature of the user is above a predefined temperature threshold. In an embodiment of the invention, the predefined temperature threshold is around 38 degrees Celsius. For example, when the temperature of the user is above 38 degrees, this can be an indication that the user is sick and carrying the COVID-19 virus. In this case, the controller will automatically activate the face shield (or a portion thereof covering the portal portion of the user's face) to close (in case it is open) and cover the portal portion of the user to reduce the risks of infecting other persons.

In an embodiment of the invention, the thermal detector is adapted to measure the temperature of another person passing in proximity of the user, and send this to the controller. When a high temperature (beyond the predefined temperature threshold) is detected, the controller automatically adjusts the face shield into the secured position (by closing the face shield or at least a portion thereof covering the portal portion of the user's face). This is to protect the user from the risk of being infected in case the person passing in proximity of the user is infectious.

In an embodiment of the invention, the controller is adapted to automatically adjusts the face shield back into the unsecured position (by opening the face shield or at least a portion thereof covering the portal portion of the user's face) in case where there is a low risk of communicating an infectious disease between the user and other persons, for example when the user does not have medical symptoms of being infected by a virus (i.e. when the user's temperature is below the temperature threshold) and in absence of other persons passing in proximity of the user.

In an embodiment of the invention, the thermal detector comprises a non-contact infrared sensor. Preferably, the non-contact infrared sensor is mounted in a position pointing at a forehead of the user when the face shield is being worn by the user, for measuring the user's temperature through the user's forehead. The non-contact infrared sensor can for example be mounted at the helmet itself or on a mounting structure coupled/part of the helmet such as a component which is designed to hold the face shield in place. At an embodiment of the invention, the infrared sensor is mounted at the internal side of the helmet such that it points at the forehead of the user when it is being worn by the user. In another embodiment, the infrared sensor line of sight projects outwardly away from the user to detect the temperature of an intruding person passing in proximity of the user (within the minimum social safety distance threshold for example).

In an embodiment of the invention, the Health & Safety Monitoring Unit comprises a social distance detector for detecting presence of another user within a predefined minimum social safety distance threshold, and wherein the controller is configured to put the face shield in the secured position (closing the face shield or at least a portion thereof covering the portal portion of the user's face) when another user is detected within the predefined minimum social safety distance threshold. This is in order to reduce the risks of passing infectious droplets or fluid between the user and other persons passing in proximity of the user within the minimum social safety distance threshold. Preferably, the social safety distance threshold is around 6 feet.

In an embodiment of the invention, the social distance detector comprises a proximity detector which can comprise at least one of an infrared sensor, an ultrasound sensor, and an optical sensor.

In an embodiment of the invention, the user is a member of a community (such as a school or a hospital or a care center) and wherein other community members are wearing a similar smart helmet, such that each smart helmet has a unique identifier configured to uniquely identify the helmet and the user/community member wearing the helmet. In an embodiment of the invention, the unique identifiers associated to the intruding persons of the user (those breaching the minimum social safety distance threshold) are obtained by the smart helmet of the user and stored inside the memory.

In an embodiment of the invention, the social distance detector comprises an ultra-wide band based distance measuring unit which is configured to measure the distance between the user and other members of the community wearing a similar smart helmet device. In an embodiment of the invention, tracking information associated to a user and all intruding persons are stored within the memory of the smart helmet of the user or an external server. This information can be referred to at any later stage to track and assess health risks of the user or other persons who have been in direct contact with the user at any given time.

In an embodiment of the invention, the Health & Safety Monitoring Unit comprises a hands-to-face detector for detecting a hand gesture of the user to touch his face, and wherein the controller is configured to secure the face shield in the secured position when the hand gesture of the user to touch his face is detected. In an embodiment of the invention, the hands-to-face detector comprises a proximity sensor mounted in a position capable of detecting presence of the user's hands in proximity of the user's face. In an embodiment of the invention, the proximity sensor is an infrared sensor. However, the proximity sensor can also be an ultra-sound sensor or any other suitable type of sensors capable of detecting presence of hands in proximity of the user's face.

In an embodiment of the invention, the proximity sensor is mounted at the helmet and positioned such that it detects presence of the user's hands when these come in proximity of the user's face (or the user's portal portion of the face).

In an embodiment of the invention, the proximity sensor is an infrared sensor mounted along the perimeter of a face opening portion of the helmet.

In another embodiment of the invention, the hands-to-face detector comprises infrared sensors positioned within a bracelet pointing towards the user's wrist and configured to detect a hands-to-face movement based on an image of the wrist captured by said infrared sensors.

In an embodiment of the invention, the helmet comprises a mounting structure for receiving the face shield unit, the Health & Safety Monitoring Unit and the controller.

In an embodiment of the invention, the user is an infant or a person with special needs. The smart helmet is configured to be worn by the user and to automatically open and close the face shield based on the health and safety conditions of the user. This is as an infant might not have the judgment and/or the speed and/or the physical capacity to close the face shield in case another person is passing in proximity or in case he has a high temperature or in case there is any other risk condition. At the same time, it will be difficult to have an infant to cover their faces for a long period of time as this becomes uncomfortable for them to breathe and communicate with others. Also, a person in special needs, such as a patient at a hospital or a handicapped person on a wheel chair or an elder person in an elderly house) might lack the judgment and/or the physical condition to open and close the face shield during the day and might feel uncomfortable or impractical to have the face shield covered during long hours.

In an embodiment of eth invention, the face shields covers all the portals of entry for this virus: the eyes, the nose, and the mouth. Moreover, the supply chain is significantly more diversified than that of face masks, so availability is much greater. Face shields are the easiest type of personal protective equipment (PPE) to make. They typically consist of just two parts: a visor that covers the face and which is usually made of plastics such as polycarbonate, propionate, acetate, polyvinyl chloride (PVC), and polyethylene terephthalate glycol (PETG); and a method of holding the visor in place, such as a headband or strap. The strap can be made of moulded plastic, 3D-printed plastic or even elastic.

As a further aspect of the invention, there is provided a smart face protective system for preventing or reducing spread of infectious diseases within a community comprising community members, the system comprising:
Smart face protective devices (SFPDs) in accordance with the various embodiments of the present invention for use respectively by the community members such that each SFPD is uniquely identified and associated to a single community member;
A SFPD Admin System in communication with the plurality of SFPDs through a wireless data network for remotely administrating the SFPDs and for receiving and processing health and safety data related to the community members as collected by the SFPDs and for outputting protective and/or corrective healthcare measures based on the collected data.

As a further aspect of the invention, there is provided a SFPD network for preventing or reducing spread of infectious diseases within a community comprising community members, the Network comprising:
N number of SFPD Groups (90A, 90B, . . . , 90N), where each SFPD Group (90A, 90B, . . . , 90N) comprises K number of SFPDs (10A, 10B, . . . , 10K);
N number of SFPD Control Devices (80A, 80B, . . . , 80N), where each SFPD Control Device (80A, 80B, . . . , 80N) is connected/configured to be connected to one SFPD Group (90A, 90B, . . . , 90N);
a SFPD Admin System connected/configured to be connected to the N SFPD Control Devices.
where N is greater than or equal to 2 and K is greater than or equal to 2

Preferably, the SFPDs of the network are smart face protective devices according to the various embodiments of the present invention.

As a further aspect of the invention, there is provided a method of preventing or reducing spread of infectious diseases within a community comprising community members, the method comprising:
dividing the community members into N groups, such that each group comprise K members;
providing the K community members with K smart face protective devices in accordance with the various embodiments of the invention;
providing N Admin Control Devices for respectively managing and controlling the N groups such that each Amin Control Device is paired and connected to K smart face protective devices associated to a given group for simultaneously managing and controlling said K smart face protective devices.

Preferably, the method further comprises providing a SFPD system in communication with the A Admin Control Devices for remotely administrating the Admin Control Devices and for receiving and processing health and safety data related to the community members as collected by the smart face protective devices and for outputting protective and/or corrective healthcare measures based on the collected data.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1 is a Smart Face Protective Device (SFPD) for preventing or reducing spread of infectious droplets and body fluids through a portal portion of the user's face in accordance with an embodiment of the present invention;

FIG. 2 is a Smart Face Protective Device (SFPD) in accordance with another embodiment of the present invention;

FIG. 3 is a Health & Safety Monitoring Unit of the SFPD for monitoring health & safety conditions in accordance with an embodiment of the present invention;

FIG. 4 illustrates a SFPD Face Shield Unit in accordance with an embodiment of the present invention;

FIG. 5 illustrates a SFPD User Interface in accordance with an embodiment of the present invention;

FIG. 6 illustrates a SFPD network with a group of SFPDs in a remote data communication with a SFPD Admin System in accordance with an embodiment of the present invention;

FIG. 7 illustrates a SFPD Network with a plurality of SFPDs divided into groups controlled by respective Admin Control Devices and an SFPD Admin System in accordance with an embodiment of the present invention; and

FIG. 8 illustrates a SFPD Control Device for simultaneously controlling/managing a group of SFPDs in accordance with an embodiment of the present invention.

### DETAILED DESCRIPTION OF THE INVENTION

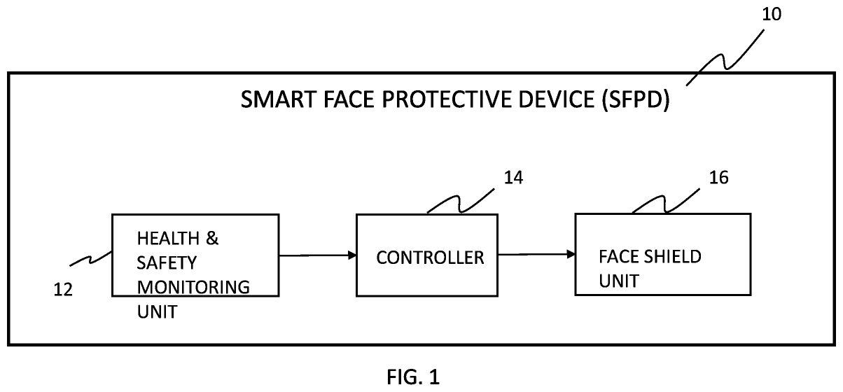

The aspects of the invention will be described in conjunction with FIGS. 1-8. In the Detailed Description, reference is made to the accompanying figures, which form a part hereof, and in which is shown by way of illustration specific embodiments in which the invention may be practiced. It is to be understood that other embodiments may be utilized and logical changes may be made without departing from the scope of the present invention. The following detailed description, therefore, is not to be taken in a limiting sense, and the scope of the present invention is defined by the appended claims.

Referring to FIG. 1, there is provided a first embodiment of a smart face protective device (SFPD) 10 for preventing or reducing spread of infectious droplets through a portal portion of a user's face. The SPFD 10 comprises a Health & Safety Monitoring Unit 12, a controller 14 and a face shield unit 16 for monitoring a health & safety condition related to a user and for automatically adjusting the face shield unit 16 between a secured position and an unsecured position based on the monitored health & safety condition related to the user. The monitoring of the health and safety condition of the user and the adjustment of the face shield unit 16 are conducted in real time. The SFPD can be a wearable device, such as helmet or a portable face shield device or another head held/wearable device, or a non-wearable device designed to be mounted to a functional article related to the user such as a wheel chair, a bed, a cubicle, a desk, a vehicle component or so on. The SFPD is preferably uniquely identified and associated to the user.

The Health & Safety Monitoring Unit 12 is in electrical communication with the controller 14 for monitoring health & safety conditions related the user (at least one condition) and, based on the monitoring, sending an electric signal reflecting the monitored health & safety conditions to the controller 14. The controller 14 is in electrical communication with the face shield unit 16. The controller 14 comprises a microprocessor and/or electronic circuit configured with predefined rules for processing the received health & safety conditions related to the user and, based on the processing, sending an electric signal controlling the face shield unit 16. The face shield unit 16 is in electrical communication with the controller 14 for automatically adjusting the face shield unit 16 between a secured position and an unsecured position based on the instructions received from the controller 14.

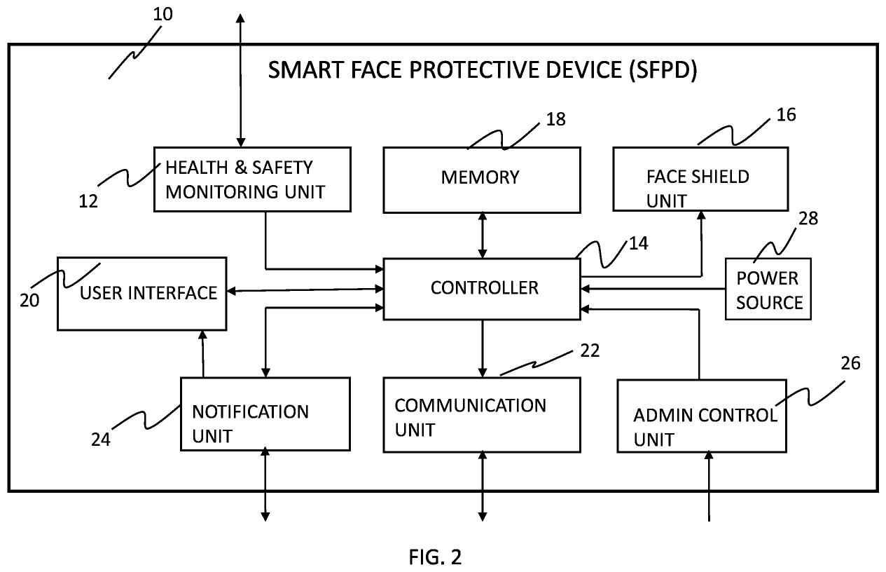

Referring to FIG. 2, in another embodiment, the smart face protective device (SFPD) 10 further comprises the following components in electrical communication with the controller 12: a memory 18, a user interface (also named input/output interface) 20, a communication unit (also called inter-device communication unit) 22, a notification unit 24, an admin control unit 26, and a power source 28.

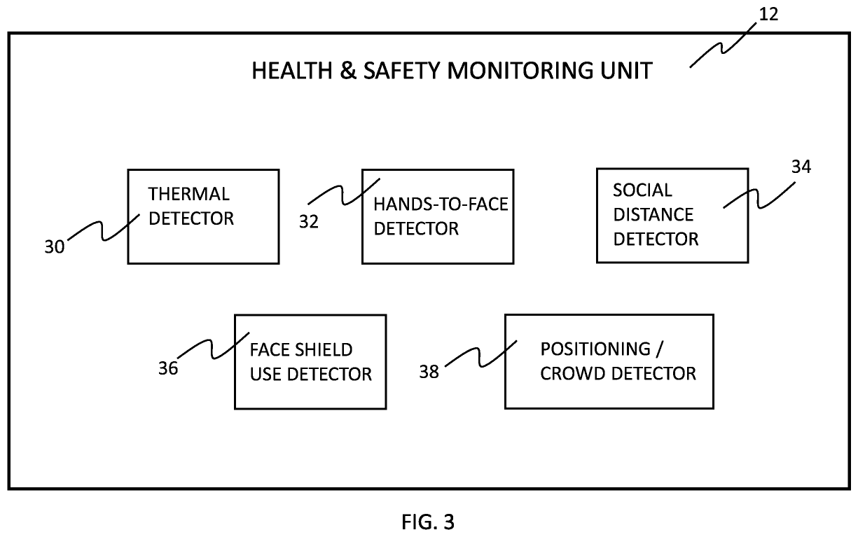

Referring to FIG. 3, the Health & Safety Monitoring Unit 12 preferably comprises a thermal detector 30 for monitoring the body temperature of the user and/or of another person. The thermal detector 30 preferably comprises a first infra-red (IR) sensor for monitoring and measuring the body temperature of the user (who is wearing/using the SFPD). The infra-red (IR) sensor can be a non-contact sensor designed to point and read the body temperature from the forehead of the user. When the SFPD comprises a helmet or another head wearable device, the IR non-contact sensor is preferably mounted to the helmet or to the head wearable device in such a manner that the IR non-contact sensor points to the forehead of the user or another member of the user's body from which the user's body temperature can be read. As the health & safety condition of the user also depends on the health conditions of persons coming into direct physical contact with the user, the thermal detector 30 is preferably configured to detect the body temperature of other persons coming into direct physical contact with the user (also called persons being in proximity of the user or intruding persons). In this case, the thermal sensor 30 preferably comprises a second thermal sensor (such as IR sensor) being configured to measure the temperature of persons coming into direct contact with the user (preferably those coming within the minimum social safety distance threshold) and sending this information to the controller 14.

The controller 14 is configured to receive the body temperature obtained by the thermal detector 30 and to determine whether the body temperature falls below or above a predefined temperature threshold (for example 38 degree Celsius) and to make a decision based on this determination. If the body temperature of the user (or the intruding person) is above the predefined temperature threshold, then the controller 14 sends an electric signal to the face shield unit 16 for automatically adjusting the face shield unit 16 into a secured position such that a barrier is established between the user's portal portion of the face and the external environment to block communication of any droplets or other fluids between the user and other persons. The detection of the body temperature by the thermal detector 30, the processing and determination of a high body temperature by the controller 14 and the automatic adjustment of the face shield unit 16 into a secured position are all conducted in real time and within a very short time period (preferably less than 1-2 seconds) in order to reduce infection risks.

The Health & Safety Monitoring Unit 12 preferably further comprises a Hands-to-Face Detector 32 for detecting a hand gesture of the user (or another person such as another child trying to reach out to the user's face) to touch the user's face. The Hands-to-Face Detector 32 preferably comprises one or more sensors (is referred to as a hands-to-face sensor for purpose of language simplification) configured to detect either a particular movement, shape or location of one or both hands which is indicative of a hand gesture to reach out to the user's face. The sensor can be a proximity sensor such as an IR sensor, an ultrasound sensor or any other suitable type of sensors. The hands-to-face sensor can be an IR sensor for detecting presence of hands in proximity of the user's face. In case the SFPD comprises a helmet or another head wearable device, the IR sensor can be mounted to the helmet/head warble device in such a way to detect presence of hands (or another object) in proximity of the user's face. The hands-to-face detector 32 can also comprise a plurality of infrared sensors positioned within a bracelet pointing towards the user's wrist and configured to detect a hands-to-face movement based on an image of the wrist captured by said infrared sensors.

The controller 14 is configured to receive data obtained by the hands-to-face detector 32 and to process this data to determine whether there is an attempt by the user (or another person) to reach out to the user's face using hands. If it is the case, then the controller 14 sends an electric signal to the face shield unit 16 for automatically and adjusting the face shield unit 16 into a secured position such that a barrier is established between the user's portal portion of the face and the external environment to block hands reaching out to the user's face. The detection of the hands-to-face gesture by the hands-to-face detector 32, the processing of the information by the controller 14 and the automatic adjustment of the face shield unit 16 into a secured position are all conducted in real time and very quickly within a very short time period (preferably less than 1-2 second) in order to block the hands before reaching out to the face.

The Health & Safety Monitoring Unit 12 preferably further comprises a Social Distance Detector 34 for detecting presence of an intruding person within a predefined minimum social safety distance threshold of the user. The social distance detector 34 preferably comprises one or more proximity sensors, such as ultra sound sensors, infra-red sensors or laser sensors to measure the distance between the user and the intruding persons. The social distance detector 34 may also use an ultra-wide band technology to measure the distance, particularly when the intruding persons are other users using similar SFPDs such that these SFPDs can communicate with each other to exchange data based on which the speed of the data communication can be measured to determine the distance between these various SFPDs.

The controller 14 is configured to automatically adjust the face shield unit 16 in the secured position when the intruding person is detected within the predefined minimum social safety distance threshold. The social safety distance threshold is normally in the range of 6 feet, however it is preferable to be set in the range of 6-10 feet to be more on the safe side. The predefined minimum social safety distance is set based on the recommendations of the world health organization for the prevention of the COVID-19 and other viruses of similar nature. The detection of an intruding person by the social distance detector 34, the processing of the information by the controller 14 and the automatic adjustment of the face shield unit 16 into a secured position are all conducted in real time and very quickly within a very short time period (preferably less than 1-2 seconds) in order put the face shield unit 16 in the secured position before the intruding person gets to a critical infectious position.

The Health & Safety Monitoring Unit 12 preferably further comprises a Face Shield Use Detector 36 for detecting and providing an indication on whether the user is wearing the SFPD 10 which is particularly useful in case the SFPD is part of a helmet structure wearable by the user (such as a child) and the wearing of the SFPD needs to be monitored by a remote administrator. The Face Shield Use Detector 36 can comprise any suitable sensor, such as a proximity sensor or an infra-red sensor which can detect whether the user is wearing the helmet with the SFPD device. The infra-red sensor can be the same infra-red sensor used to measure the body temperature of the user. In fact, if the infrared sensor reading is below a human body temperature (for example 37 degrees and below), this is indicative of the absence of human body which can be interpreted as a non-use of the SFPD helmet device by the user. Other suitable sensors can be used such as a switch button which is activated when the helmet is wore and deactivated otherwise. The controller 14 receives an indication from the Face Shield Use Detector 36 on whether the face shield is being used by the user. The controller 14 can store the information in a memory 18 and/or can notify a remote administrator of the same depending on the application and configuration of the SFPD 10. For example, in the case of school children required to wear helmet based SFPDs, a school administration can monitor weather children are wearing their helmet SFPDs, and when a given child is not wearing the helmet SFPD, the Face Shield use Detector 36 detects that the helmet based SFPD is not being wore by the child and this information is fed to the controller 14 which sends a notification to an admin control device 80 (held by the school administration) through a notification unit 24.

The Health & Safety Monitoring Unit 12 preferably further comprises a positioning/crowd detector 38 for tracking and recording the location of the user. The positioning/crowd detector 38 preferably comprises a global positioning system (GPS) device or another positioning technology to track and locate the geographical location of the user, including in an indoor environment. In an exemplary embodiment of the invention, SFPDs 10 are provided to one or more groups of users within a community controlled by a community administration (for example children at a school controlled by a school administration or patients at a hospital controlled by a hospital administration) such that each user is associated to a respective SFPD 10. The locations of the users are tracked through the positioning/crowd detector 38 of their respective SFPDs which obtains the positions of the users in real time and sends it to a crowd detection module within an SFPD administration system 40 which maps the real time positions of the users and generate a crowd map showing the crowd densities with a highlight on any overcrowded area to be avoided by other users or to be inspected by the community administration.

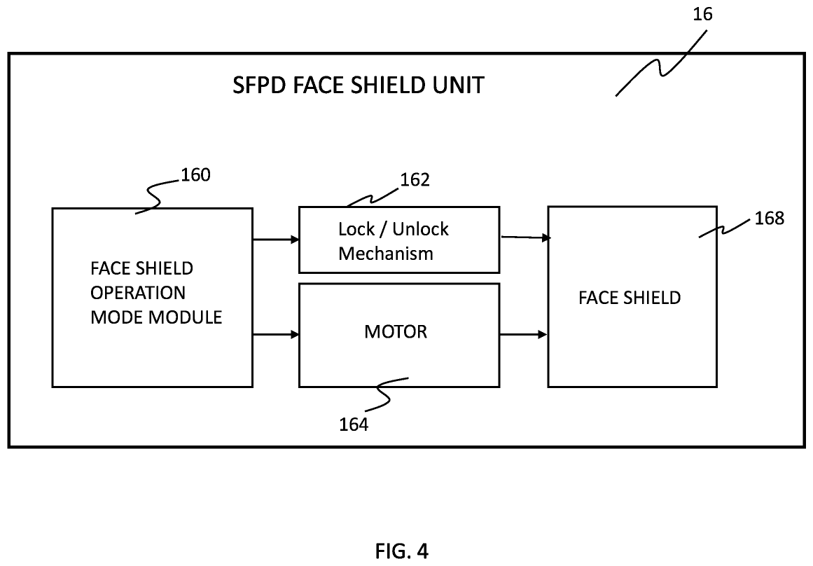

Referring to FIG. 4, the Face Shield Unit 16 comprises a face shield operation mode module 160 for setting the operation mode of the face shield among a manual operation (where the face shield is manually operated by the user either to be in the secure position or in the insecure position), a secure mode where the face shield is locked in the secure position (meaning the face shield is secured/closed and locked in that position), an insecure mode where the face shield is locked in the insecure position (meaning the face shield is insecure/opened and locked in that position), an automatic mode (where the face shield is automatically operated and adjusted by the controller based on the health and safety condition of the user). The face shield operation mode module 160 obtains the instruction on the operation mode from the controller 14.

The face shield unit 16 further comprises a lock/unlock mechanism 162 and a motor 164 connected to the face shield operation mode module 160 and to the face shield 168. The lock/unlock mechanism comprises a mechanical or an electromagnetic clutch for locking the face shield 168 in a given position (insecure or secure position) which is also adapted to be deactivated to return the face shield in an unlocked position. The lock/unlock mechanism 162 obtains the lock/unlock instructions from the face shield operation mode module 160. The motor 164 is preferably an electrical motor connected to the face shield 168 for automatically adjusting the face shield between a secure position (for example by closing the face shield) and an insecure position (for example by opening the face shield). When the face shield operation mode is set to be automatic, the motor 164 automatically adjusts the face shield 168 between a secure and insecure position depending on the health and safety condition of the user as instructed by the controller 14. When the face shield operation mode is set to be manual, the motor 164 is receptive to a manual instruction by the user or by the administrator to either put the face shield in a secure position (by closing the face shield for example) or to put it in the insecure position (by opening the face shield for example).

hi an exemplary embodiment, SFPDs 10 are used by a group of children at school class where each child is provided by a corresponding SFPD as a personal protective equipment. The teacher (as administrator of the SFPDs) can lock the face shields 168 of the SFPDs 10 in the secure position where the all the face shields 168 of the SFPDs associated with the children will be secured/closed and locked in that position in the sense that the children will not be able to insecure/open the face shields manually. This mode can be set when the children are in a group activity in physical proximity to each other for example. The face shields 168 can also be locked in an insecure position (all shields open) when the kids are seated on their distant desks during a lecture for example. The teacher can also chose to set the face shields 168 in an automatic mode where the face shields will open and close based the monitoring of the health and safety conditions related to the children. This mode can be set when the children are in a free activity outside the classroom for example.

The SFPD 10 comprises a memory 18 is in data communication with the controller 14 for storing and providing reading access to a unique identifier enabling to uniquely identify the user and/or the SFPD 10 associated thereto. The memory 18 is also configured to store information about health & safety conditions associated to the user such as body temperatures of the user, the identity and the body temperatures of persons with whom the user was in direct contact during a specific period of time (also called the intruding persons), the distances between the user and the intruding persons with an indication of any breach of the minimum social safety distance threshold by the intruding persons, the locations of the user during a specific period of time or during a specific incident for example while a high risk intruding person got into direct contact with the user, the crowd density in the various locations of the user during a specific period of time and an indication on whether the user was in a highly crowded area which may be indicative of a high risk, a count of the hands-to-face gestures/block actions by the face shield and any failures to block the hands by the SFPD, and so on. The memory 18 can store very valuable health and safety data related to the user (which may be in relation to the user him/herself or to the intruding persons) which may be tracked/accessed at a time in the future to analyze and determine any health and safety vulnerability or breach or to track persons who were in direct physical contact with the user.

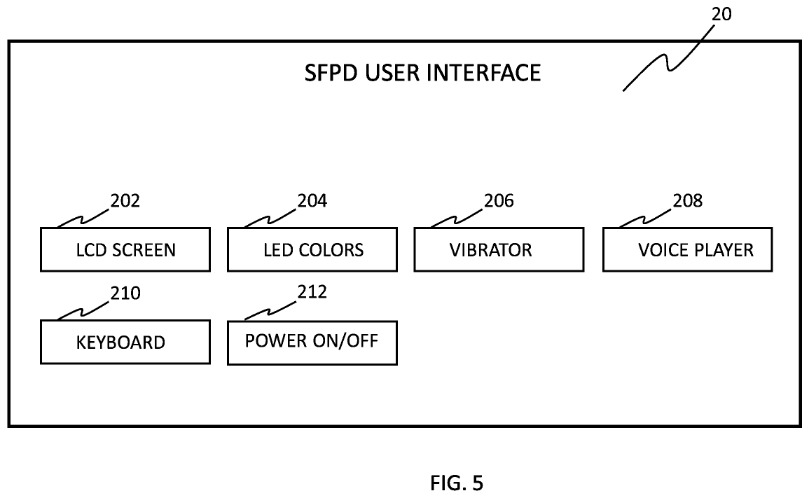

Referring to FIG. 5, the SFPD 10 comprises a user interface 20 (also called user input/output unit) comprising a LCD screen 202, LED colors 204, a vibrator 206, a voice player 208, a keyboard 210 and a power on/of 212. The user interface 20 is in data communication with the controller 12 for inputting/outputting data between the controller 12 and the user interface 20 to/from the user and surrounding persons. For example, the controller 12 can activate the vibrator 206 to alert the user when an intruding person is approaching in proximity of the user in breach of the social safety distance. Based on the alert, the user can stop, change direction or take any other precautionary measure. The controller 12 can also activate the voice player 208 to play a voice message to the user or to the intruding person alerting about the breach. The voice message can also be customized based on the identity of the intruding person. For example, in case where the SFPDs are used by school kids, a first SFPD associated to a first kid obtains the identity of an intruding kid by way of data communication between the first SFPD (associated to the first kid) and a second SFPD associated to the intruding kid. The identity of the intruding kid is stored inside the memory 18 of the second SFPD and transmitted to the first SFPD through the inter-device communication unit 22. In this case, the voice message played by the first SFPD can be customized to mention the name of the intruding kid, such as “Maria, please keep distance”. The voice message when customized to mention the name of the intruding person will be perceived as friendlier and less intrusive particularly for kids. The LCD screen 202 can be used to display a message which can be updated on a real time basis. The LED colors 204 can be used to identify groups of users. For example, school kids can be divided in a various groups where each group is given a specific color code which is displayed through the LED Colors 204. This is as social distancing is becoming more and more important at schools, should administration might need to divide kids into different groups each of which have a different time schedule for using public areas such as the playgrounds, the library and the bathrooms. If a group of kids identified by a “blue” color for example are scheduled to play at the playground at a specific time period, it will be easy to identify through the color code any breach of the schedule by other kids not belonging to the same group. If a kid with “red” color appears in the playground, this kid will be easily spotted as breaching the social distancing/scheduling rule. The keyboard 210 can be used to set/change some configurations of the SFPD such as the user identity, the temperature threshold, the minimum social safety distance threshold, the color codes, and so on. The power on/off 212 will enable to switch on and off the SFPD 10.

The SFPD 10 comprises a notification unit 24 in electrical communication with the controller 14 configured to notify a health/safety risk condition such as a high temperature above the predefined temperature threshold or a breach of the minimum social safety distance. The notification unit is configured to notify the user or other users through a user interface 20 such as a screen, a led, a voice message, or a vibrator. In an embodiment of the invention, the notification unit is configured to be connected to a remote administration control device 80 through a data network (such as WIFI) to notify an administrator (such as a teacher or school director) of presence of a health/safety risk associated to the user.

The SFPD 10 comprises s communication unit 22 (also called inter-device communication unit) in electrical communication with the controller 14 and with external remote devices through a data network. In an embodiment of the invention, the inter-device communication units 22 associated to the various face protective devices 10 are configured to exchange health/safety data relative to their respective users (for example the identity of the intruding persons passing within the social safety distance threshold, and the temperature of the intruding users) and to store this data inside the memory 18 of their respective SFPDs 10.

In an embodiment of the invention, the data exchanged by a SFPD 10 comprises an indication of any health/safety risk associated to its respective user (for example an indication of high temperature of the user, or a measure of the temperature itself), and wherein the SFPD 10 automatically adjusts the face shield unit 16 in the secured position (for example by closing the face shield 168) when/if the temperature of the intruding person as obtained by the communication unit 22 is above the predefined temperature threshold. Preferably, the identity of the intruding user (or associated face protective device) is stored inside the memory 18 of the user's face protective device 10 which can be retrieved at any future time for tracking and identification purposes of infected persons.

The Health & Safety Monitoring Unit 12 can comprise an external component (system or device) located remote from the SFPD 10. In this case, the communication between the controller 14 and the external component/device of the Health & Safety Monitoring Unit 12 is a remote wireless communication (through a data network such as WIFI or Bluetooth or any other suitable wireless communication technology) conducted through the communication unit 22 of the SFPD 10.

In an exemplary embodiment, the external component of the Health & Safety Monitoring Unit 12 comprises an identification system configured to identify the user. The identification system can for example be an RFID system wherein the RFID reader can be located at the remote (external) component of the Health & Safety Monitoring Unit 12 and the RFID tag is located at the SFPD 10. The external component of the Health & Safety Monitoring Unit 12 identifies the user, monitors his body temperature and sends a signal to the SFPD 10 of the user (through the controller) in order to automatically adjust the shield in the secured position when the temperature of the user is beyond the predefined temperature threshold. In another embodiment, the external component comprises the thermal detector of the Health & Safety Monitoring Unit 12 which screens the body temperature of other persons passing or being located in proximity of the user (the intruding persons), and sends a signal to the SFPD 10 of the user (through the communication unit 22 connected to the controller 14) in order to automatically adjust the face shield 168 in the secured position when the temperature of an intruding person is beyond the predefined temperature threshold.

The SFPD 10 comprises an admin control unit 26 in electrical communication with the controller 14 and with a remote admin control device 80 through a data network. The admin control unit 26 is adapted to receive remote instructions from the remote admin control device 80 with respect to the operation and control of the SFPD 10. In an embodiment, the admin control unit 26 receives an activation and deactivation signals to respectively activate and deactivate the SFPD 10. The admin control unit 26 is adapted to activate/deactivate the SFPD 10 based on the received signal from the admin control device 80. In another embodiment, the admin control unit 26 receives a notification or a message from the administrator which can be communicated to the user or surroundings via the user interface 20. The message or notification is processed by the controller 14 and then communicated to the user interface 20. In another embodiment, the admin control unit 26 receives instructions from the admin control device 80 with respect to the operation mode of the face shield unit 16 (for example, manual, automatic, locked secured or locked insecure). These instructions are sent to the controller 14 for processing, where the controller 14 controls the SFPD face shield unit 16 based on these instructions. In another embodiment, the admin control unit 26 comprises a pairing module adapted to pair the SFPD 10 with a given admin control device 80. In one example, the pairing is conducting through frequency alignment/pairing between the two devices 10 and 80. In another embodiment, the admin control unit 26 receives a labelling signal to label the SFPD 10 for example through the allocation of a given color code to the LED colors 24 of the user interface 20. This is to be able to identify users as part of a given group (labelled by the given color code) among various groups.

The SFPD 10 comprises a power source 28 which is preferably a rechargeable battery and adapted to supply power to all the electrical components of the SFPD.

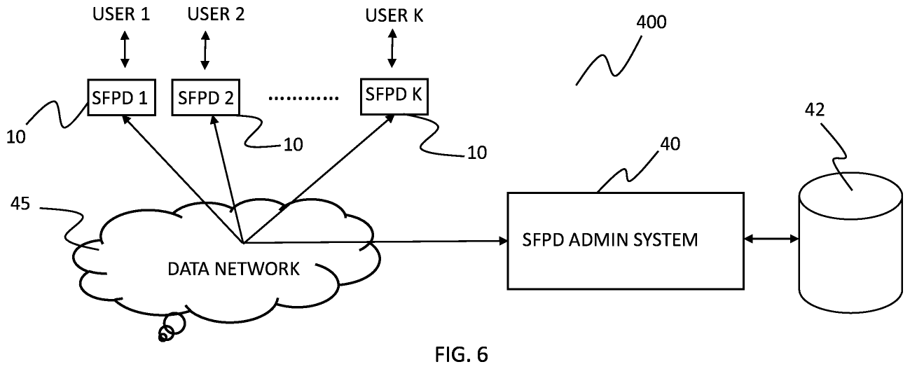

As another aspect of the invention, referring to FIG. 6, there is provided a SFPD network 400 comprising a plurality of smart face protective devices (SFPDs) 10 associated to respective users within a given community (such as a school or a hospital), a SFPD Admin System 40 connected to the SPFDs 10 through a data network 45, and a SFPD Admin Database 42 connected to the SFPD Admin System 40 for storing data related to health and safety conditions of the users as received from the SFPDs 10 which enables the tracking and analysis of health and safety records in connection with the users. The SFPD Admin System 40 is configured to obtain the data stored within the memory 18 of the SFPDs 10, to compile and analyze the obtained data based on a predefined health data management algorithm and to output precautionary/proactive health measures and/or corrective/reactive health measures in connection with the users. For example, if a particular person is diagnosed COVID-19 positive (infected person) in a particular period, it will be possible through the stored records in the database 42 and the SFPD admin system 40 to identify the other persons at risk and take measures to reach out and test these persons at risk. This can be conducted by for example tracking back all others persons within the community who have been into direct physical contact with this infected person.

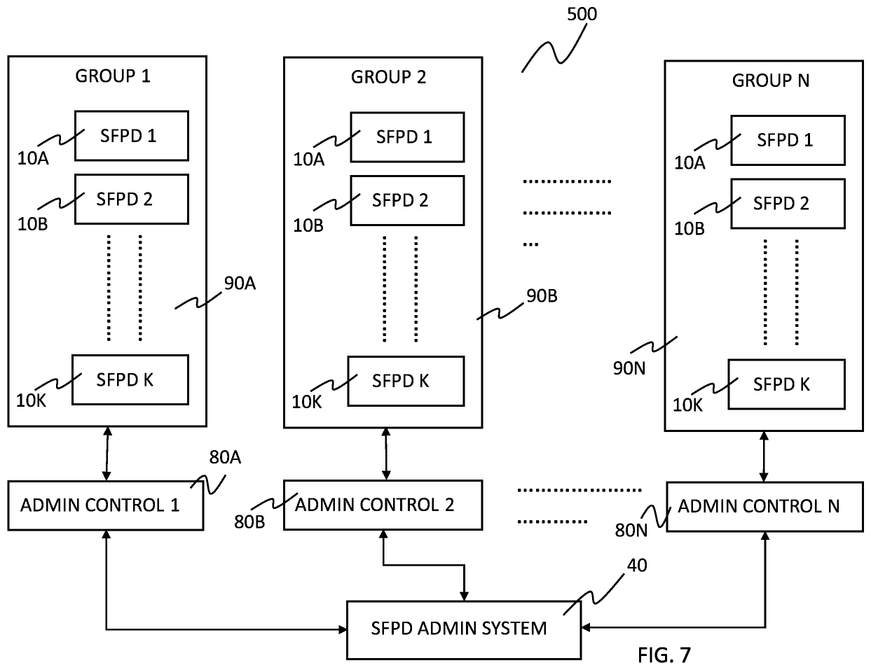

As a further aspect of the invention, referring to FIG. 7, there is provided a SFPD Network 500 comprising:
N number of SFPD Groups (90A, 90B, . . . , 90N), where each SFPD Group (90A, 90B, . . . , 90N) comprises K number of SFPDs (10A, 10B, . . . , 10K);
N number of SFPD Control Devices (80A, 80B, . . . , 80N), where each SFPD Control Device (80A, 80B, . . . , 80N) is connected/configured to be connected to one SFPD Group (90A, 90B, . . . , 90N);
a SFPD Admin System 40 connected/configured to be connected to the N SFPD Control Devices.
where N≥1 and K≥1; however, preferably N≥2 and K≥2; and more preferably N≥3 and 3≥K≥5 (for minimizing grouping between individuals and increasing social distancing);

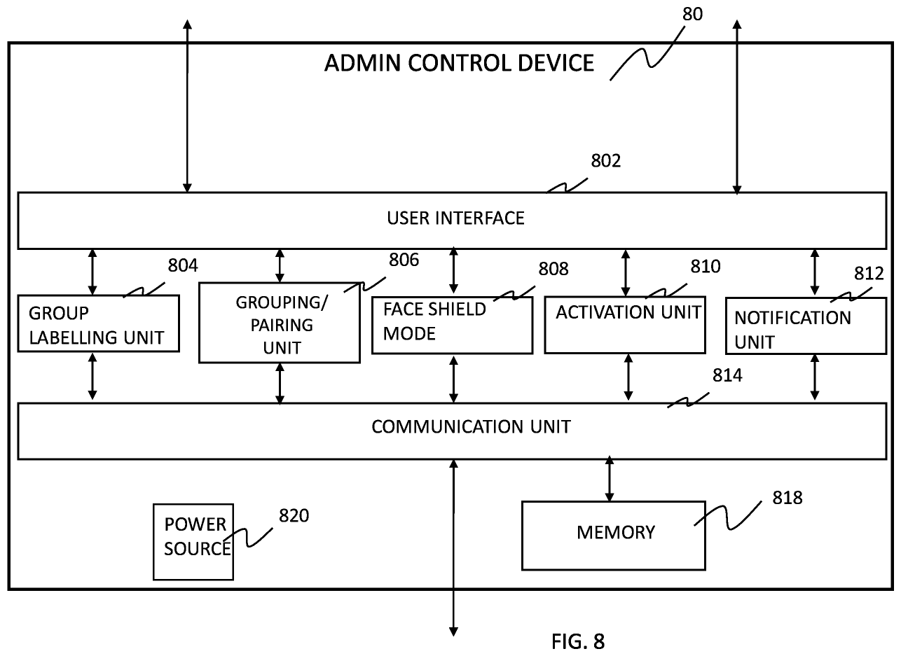

Referring to FIG. 8, the Admin Control Device 80 comprises a user interface 802, a group labelling unit 804, a grouping/pairing unit 806, a face shield mode 808, an activation unit 810, a notification unit 812 and a communication unit 814.

The user interface 802 is connected to the grouping/pairing unit 806 for enabling the identification/selection of the K SFPDs (10A, 10B, . . . 10K) to be paired/connected to the Admin Control Device 80. This identification/selection can be conducted by entering the unique identifiers associated to the K SFPDs (10A, 10B, . . . 10K) or through a pairing technique between the K SFPDs (10A, 10B, . . . 10K) and the Admin Control Device 80. The user interface 802 and the grouping/pairing unit 806 are configured to enable the selection/identification of the K SFPDs (10A, 10B, . . . 10K) which are to be paired to that specific Admin Control Device 80.

This process is repeated for each one of the N SFPD Groups (90A, 90B, . . . , 90N) for enabling the pairing between each one of the N Admin Control Devices (80A, 80B, . . . , 80N) associated to a specific group among the N SFPD Groups and its respective K SFPDs (10A, 10B, . . . , 10K) such that each Admin Control Device among the Admin Control Devices (80A, 80B, . . . , 80N) is paired to K SFPDs (10A, 10B, . . . 10K) and this is repeatedly for each and every one of the N SFPD Groups.

For example, a school of 100 children can be divided in 20 groups, each group comprising 5 kids. The kids of the school are provided with SFPDs such that each kid has a corresponding SFPD uniquely identified under his name. The total number of SFPDs is equal to the total number of kids which is 100. The total number of SFPD Groups formed in this case is the total number of groups formed with the kids which is N=20. The total number of Admin Control Devices in this case is N=20. The total number of SFPDs selected/identified under each group in this case is K=5. In this example, there is provided 20 Admin Control Devices 80 where each Admin Control Device 80 is configured to be connected/paired to 5 SFPDs respectively for each one of the 20 SFPD Groups formed for the 20 Groups of Kids. Each Admin Control Device (80A, 80B, . . . , 81K) enables the identification/selection/pairing of the K SFPDs associated to a given SFPD Group among the N Groups, such that the K SFPDs in that given group can communicate exclusively with that specific Admin Control device 80 to which they have been paired. Any message or notification transmitted from a SFPD 10 will be received by the corresponding Admin Control Device 80 to which it has been paired.

The grouping/pairing enables one Admin Control Device (for example Admin Control Device 80B) to simultaneously operate and control the K SFPDs paired with that specific Admin Control Device 80B. For example, the administrator of the Admin Control Device 80B can provide the green labelling code to his group of kids using the Admin Control Device 80B which will automatically be communicated to/and reflected on the SFPDs paired to that specific Admin control Device 80B. This simultaneous control/management of the SFPDs is applied to all other functions and features provided by the Admin Control Device 80 for the management/control/administration of the SFPD 10 which includes the group labelling provided by the group labelling unit 804, the face shield operation mode which is provided by the face shield mode module 808, the activation/deactivation of the SFPD which is provided under the activation unit 810, the notification of the users through alert communication/notifications which is provided under notification unit 812.

The user interface 802 is connected to the group labelling unit 804 for enabling the unique labelling of the SFPD 10 according to a specific labelling code or name. For example, this can be done through a color code which is communicated to the SFPDs and displayed by the LED Colors 204 of the SFPD 10. In the example set out above, the 10 groups can be given 10 different color codes where each SFPD 10 among the 100 SFPDs would display by its respective LED Colors 204 the color related to the group to which it belongs. In this example, the SFPDs in a particular SFPD Group 1, 2, . . . , N will have a color code 1, 2, . . . N respectively. The labelling can also be done through name appellation, such as “lion class” or “tiger class” or “elephant class” which is displayed on the LCD screen 202 of the SFPD user interface 20.

The user interface 802 is connected to the face shield mode module 808 to enable the administrator to select the operation mode of the face shield unit 16 (manual, automatic, locked secured or locked insecure). This mode is communicated to the SFPDs 10 through the communication unit 814 and used to operate the SFPD Face Shield Units 16.

The user interface 802 is connected to the activation/deactivation unit 810 to enable the administrator to activate/deactivate (turn on/turn off) the SFPDs. This mode is communicated to the SFPDs 10 through the communication unit 814.

The user interface 802 is connected to the notification unit 812 for receiving any message or alert from the SFPD 10 (such as an alert of high temperature). This notification is communicated to the administrator through the user interface 802.

The Admin Control Device 80 is configured to receive the health and safety user related data stored inside the memory 18 of the corresponding SFPD 10 to which it is paired to store this information inside the memory 818 of the Admin control Device 80.

The Admin Control Device 80 also comprises a power source 820, preferably a rechargeable battery, for providing power supply to the various electronic components of the device 80

The Admin Control Device 80 is configured to communicate with the SFPD 10 through the Admin Control Unit 26 which is configured to receive the various signals from the Admin Control Device 80 and convert them into signals comprehensive by the controller 14 of the SFPD 10.

The N Admin Control Devices (80A, 80B, . . . , 80N) are connected/configured to be connected to the SFPD Admin System 40 through a data network (preferably wireless) for transmitting the health and safety user related data to the SFPD Admin System 40 for storage at the database 42 and further processing and analysis.

The SFPD Admin System 40 is also configured to control/manage the N Admin Control Devices (80A, 80B, . . . , 80N) for controlling the SFPDs. According to this embodiment, all the instructions received through the user interface 802 for the operation of the various modules and units 804, 806, 808, 810 and 812 are rather received from the SFPD Admin System 40 through the communication unit 14.

Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1. A smart face protective device (SFPD) to be used by a user for preventing or reducing spread of infectious droplets through a portal portion of the user's face, where the face protective device is configured to be automatically adjusted while being in use between a secured position and an unsecured position based on an electric signal associated to a monitored health and safety condition of the user.

2. The smart face protective device as claimed in claim 1, wherein the secured position comprises a position in which the portal portion of the user's face is isolated from the external environment for blocking spread of splashes and sprays between the user and others through said portal portion and for blocking the user's hands from reaching to said portal portion of the face.

3. The smart face protective device as claimed in claim 1, wherein the unsecured position comprises a position in which at least a part of the portal portion is not in a secured position.

4. The smart face protective device as claimed in claim 1, wherein the portal portion of the face comprises the eyes, nose and mouth.

5. The smart face protective device as claimed in claim 1, comprising:
a controller in communication with a Health & Safety Monitoring Unit for receiving the electric signal associated to the health and safety condition of the user in real time while the Health & Safety Monitoring Unit is in operation for monitoring the health and safety condition of the user.

6. The smart face protective device as claimed in claim 5, wherein the Health & Safety Monitoring Unit comprises a thermal detector for monitoring a body temperature of the user and wherein the controller is configured to automatically adjust the face protective device in the secured position when the body temperature of the user is above a predefined temperature threshold.

7. The smart face protective device as claimed in claim 6, wherein the Health & Safety Monitoring Unit further comprises a social distance detector for detecting presence of an intruding person within a predefined minimum social safety distance threshold of the user, and wherein the controller is configured to automatically adjust the face protective device in the secured position when the intruding person is detected within the predefined minimum social safety distance threshold.

8. The face protective device as claimed in claim 1, comprising a shield configured to be automatically closed for securely shielding the portal portion of the user's face when the face protective device is in the secured position, and to be open when the face protective device is in the unsecured positon.

9. The smart face protective device as claimed in claim 5, wherein the Health & Safety Monitoring Unit is an external Health & Safety Monitoring Unit located remote from the smart face protective device and wherein the communication between the controller and the Health & Safety Monitoring Unit is a remote wireless communication.

10. The face protective device as claimed in claim 1, wherein the face protective device is a portable device configured to be carried or worn by the user, the face protective device comprising a mounting structure configured to be carried or worn by the user, and a transparent face shield coupled to the mounting structure and configured to be automatically adjusted in the secured and unsecured positions.

11. The face protective device as claimed in claim 1, wherein the face protective device is a non-portable device configured to be mechanically coupled to a fixed structure selected from at least one of as a cubicle, a desk, a bed, a wheelchair, and an internal component of a vehicle, or to be used in an indoor environment, and wherein the indoor environment is an education institution, an elderly care center or a hospital.

12. A smart helmet device to be worn by a user for preventing or reducing risks of spreading infectious droplets through a portal portion of the user's face, the smart helmet device comprising:
a face shield unit comprising a motor and a face shield;
a Health & Safety Monitoring Unit for monitoring a health and safety condition of a user and outputting an electric signal with an indication of the health and safety condition of the user;
a controller in communication with the Health & Safety Monitoring Unit and the face shield unit for receiving the electric signal associated to the monitored health and safety condition of the user and automatically adjusting the face shield between a secured position and an unsecured position based thereon.

13. The smart helmet device as claimed in claim 12, wherein the secured position comprises a position in which the portal portion of the user's face is isolated from the external environment for blocking spread of splashes and sprays between the user and others through said portal portion and for blocking the user's hands from reaching to said portal portion of the face, and the unsecured position comprises a position in which at least a part of the portal portion is not in a secured position.

14. The smart helmet device as claimed in claim 12, wherein the Health & Safety Monitoring Unit comprises a thermal detector for monitoring a body temperature of the user and a social distance detector for detecting presence of another user within a predefined minimum social safety distance threshold, wherein the controller is configured to put the face shield in the secured position when the body temperature of the user is above a predefined temperature threshold and when another user is detected within the predefined minimum social safety distance threshold.

15. A smart face protective system for preventing or reducing spread of infectious diseases within a community comprising community members, the system comprising:
smart helmet devices as claimed in claim 12 for use respectively by the community members such that each smart helmet device is uniquely identified and associated to a single community member;
an SFPD Admin System in communication with the plurality of smart helmet devices through a wireless data network for remotely administrating the smart helmet devices and for receiving and processing health and safety data related to the community members as collected by the smart helmet devices and for outputting protective and/or corrective healthcare measures based on the collected data.

16. A smart face protective system for preventing or reducing spread of infectious diseases within a community comprising community members, the system comprising:
smart face protective devices (SFPDs) as claimed in claim 1 for use respectively by the community members such that each SFPD is uniquely identified and associated to a single community member;
an SFPD Admin System in communication with the plurality of SFPDs through a wireless data network for remotely administrating the SFPDs and for receiving and processing health and safety data related to the community members as collected by the SFPDs and for outputting protective and/or corrective healthcare measures based on the collected data.

17. An SFPD Network comprising:
N number of SFPD Groups (90A, 90B, . . . , 90N), where each SFPD Group (90A, 90B, . . . , 90N) comprises K number of SFPDs (10A, 10B, . . . , 10K);
N number of SFPD Control Devices (80A, 80B, . . . , 80N), where each SFPD Control Device (80A, 80B, . . . , 80N) is connected/configured to be connected to one SFPD Group (90A, 90B, . . . , 90N);
an SFPD Admin System connected/configured to be connected to the N SFPD Control Devices, where N≥2 and K≥2.

18. The SFPD network as claimed in claim 17, wherein the SFPDs is a smart face protective device or a smart helmet device.

19. A method of preventing or reducing spread of infectious diseases within a community comprising community members, the method comprising:
dividing the community members into N groups, such that each group comprise K members;
providing the K community members with K smart face protective devices in accordance with claim 1;
providing N Admin Control Devices for respectively managing and controlling the N groups such that each Amin Control Device is paired and connected to K smart face protective devices associated to a given group for simultaneously managing and controlling said K smart face protective devices.

20. The method of preventing or reducing spread of infectious diseases of claim 19, further comprising providing a SFPD system in communication with the A Admin Control Devices for remotely administrating the Admin Control Devices and for receiving and processing health and safety data related to the community members as collected by the smart face protective devices and for outputting protective and/or corrective healthcare measures based on the collected data.
